(19) United States
(12) Patent Application Publication
(10) Pub. No.: US 2023/0149268 A1
(43) Pub. Date: May 18, 2023

(54) MULTI-PHASE COSMETIC COMPOSITION COMPRISING AT LEAST TWO IMMISCIBLE OILY PHASES

(71) Applicant: CHANEL PARFUMS BEAUTE, NEUILLY SUR SEINE (FR)

(72) Inventors: Irina CHICU, PANTIN CEDEX (FR); Odile FOURNIER, PANTIN CEDEX (FR)

(73) Assignee: CHANEL PARFUMS BEAUTE, NEUILLY SUR SEINE (FR)

(21) Appl. No.: 17/916,674

(22) PCT Filed: Apr. 13, 2021
(86) PCT No.: PCT/EP2021/059478

§ 371 Date: Oct. 3, 2022

(30) Foreign Application Priority Data
Apr. 14, 2020 (WO) 20315162.6

(51) Int. Cl. A61K 8/03; A61Q 1/14; A61Q 19/08

(52) U.S. Cl. CPC A61K 8/03; A61Q 1/14; A61Q 19/08; A61K 2800/33; A61K 2800/28; A61K 2800/5922

(57) ABSTRACT

A multiphase cosmetic composition that includes two immiscible oily phases defined by specific solubility parameters at room temperature. Also, the use the multiphase cosmetic composition in the cosmetic field or the dermatological field, in particular for removing makeup from, cleansing, and/or caring for the skin, the eyes, and/or the skin appendages.

This invention relates to a multiphase cosmetic composition comprising two immiscible oily phases defined by specific solubility parameters at room temperature, and their use in the cosmetic or dermatological field, and in particular for removing makeup from, cleansing, and/or caring for the skin, eyes, and/or skin appendages.

### TECHNICAL FIELD

Cosmetic compositions composed of several distinct phases, generally at least one aqueous phase and at least one oily phase, in which said phases are separated and not emulsified into one another at rest, are usually referred to by the term “multiphase compositions”. They are distinguished from emulsions by the fact that at rest, the phases are separate instead of one being emulsified into the other. The use of these multiphase compositions requires mixing beforehand in order to form a homogeneous emulsified mixture enabling simultaneous application of all ingredients contained in these phases. At rest, said phases must separate quickly and return to their initial state, this phenomenon being better known by the term “phase separation”. The advantage of this phase separation is mainly visual: the products obtained are uncommon in this field and come in the form of several preferably transparent phases, their appearance is playful and attractive to the consumer.

A rapid phase separation (or unmixing) of the two phases after their use constitutes one of the desired qualities of two-phase compositions. Indeed, obtaining a rapid phase separation is desirable for various reasons, in particular because poor separation of the two phases is perceived by users as being unattractive.

Conventional multiphase compositions are generally two-phase compositions composed of an aqueous phase and an oily phase. Such compositions comprising a single oily phase are not optimal in terms of sensory properties. Although a refreshing effect is generally obtained by the presence of the aqueous phase, these compositions are often uncomfortable and may pull on the skin. Furthermore, the addition of cosmetic active ingredients is determined by the polarity of the phases used. In conventional two-phase compositions, it is therefore possible to introduce active agents soluble in the polar phase and active agents soluble in the selected oily phase, but in the absence of an intermediate phase, an entire category of active ingredients are excluded.

In addition, with conventional two-phase compositions, an emulsion is not necessarily formed after shaking or stirring the composition, which results in unsatisfactory makeup removal, and leaves a greasy residue on the skin or keratinous appendage.

It has therefore been attempted in the past to formulate two-phase compositions consisting of two distinct immiscible phases, which, after shaking or stirring and extemporaneous and ephemeral formation of the emulsion, exhibit a rapid phase separation into two phases, without foam formation. After prior shaking or stirring, such two-phase compositions also form an ephemeral emulsion enabling application of the composition and effective makeup removal, and do so without forming a greasy residue on the skin or the keratinous appendage.

A composition of this type has for example been described in application FR-A-3049459 by the implementation of a very specific emulsifying system, comprising an inulin derivative, a lysophospholipid, a poloxamer, a polypropylene glycol buteth, and a derivative of hydrogenated castor oil.

However, that document does not teach how to achieve a multiphase composition, in particular a three-phase composition, comprising at least two immiscible oily phases. In addition, the use of surfactants is likely to cause intolerance issues, in particular in subjects with sensitive skin, which it is desirable to avoid for cosmetic products such as care products.

Finally, there is an increasing desire to reduce the use of raw materials of synthetic origin such as silicones, fluorinated derivatives, polyethylene, or polypropylene glycols, due to the negative impact of their synthesis on the environment. However, these raw materials are often essential to providing the sensory properties of comfort and softness, or to enable separation of the phases in multiphase products.

The search is therefore ongoing for a multiphase formulation comprising at least two immiscible oily phases, presenting a very clear phase separation without requiring the use of surfactants, and providing good sensory properties in terms of softness, comfort, and tolerance.

The present invention thus relates to a multiphase cosmetic composition comprising:
a first polar oily phase characterized by a surface tension greater than 30 mN/m, and having the following solubility parameters at room temperature: δₚ of between 6 and 7.8 and δₕ of between 3.7 and 10.1,
a second apolar oily phase, immiscible with the first oily phase, characterized by a surface tension of less than 28 mN/m, and having different δₚ and δₕ solubility parameters at room temperature than those of the polar solvent used in the first oily phase, said composition being surfactant-free.

In particular, an object of this invention is a multiphase cosmetic composition comprising:
a first polar oily phase comprising dipropylene glycol dibenzoate, propylene glycol dibenzoate, triethyl citrate, castor oil, or a mixture thereof, optionally combined with one or more moderately polar oils such as argan oil, meadowfoam seed oil, jojoba oil, or a mixture thereof,
a second apolar oily phase, immiscible with the first oily phase, comprising hydrocarbons, polymers obtained from a hydrocarbon monomer, or a mixture thereof, said hydrocarbons or polymers obtained from a hydrocarbon monomer preferably being selected among isohexadecane, isododecane, undecane, tridecane, polyisobutene, squalane, or a mixture thereof, said second oily phase optionally being combined with one or more moderately polar oils such as argan oil, meadowfoam seed oil, jojoba oil, or a mixture thereof, said composition being surfactant-free.

“Surfactant-free” is understood to mean, within the meaning of this application, that the composition comprises less than 2% by weight of surfactants, preferably less than 1% by weight, more preferably less than 0.5% by weight.

The patent holder has indeed noticed that, in addition to the identified intolerance problems related to the use of surfactants in multiphase products, certain surfactants such as alkyl polyglucosides give the composition an undesired whitening effect during its application, due to the “soaping” phenomenon. The system developed in this invention therefore also makes it possible to avoid whitening upon application.

The cosmetic composition according to the invention is composed of at least two distinct oily phases, is emulsified easily by shaking or stirring, and easily separates into phases after the shaking or stirring stops.

The composition according to the invention is preferably liquid and intended for topical application.

The invention also relates to a skin care product comprising such a composition, a skin care process consisting of applying such a composition to the skin, and the use of such a composition in the cosmetic or dermatological field, and in particular for removing makeup from, cleaning, and/or caring for the skin, eyes, and/or skin appendages.

First Oily Phase

The composition according to the invention makes use of a first polar oily phase characterized by a surface tension greater than 30 mN/m, preferably greater than 35 mN/m, more preferably greater than 40 mN/m, and having the following solubility parameters at room temperature: δₚ of between 6 and 7.8 and δₕ of between 3.7 and 10.1.

“Room temperature” is understood to mean a temperature of approximately 25° C.

In particular, the first polar oily phase comprises one or more polar solvents.

The polar solvents used in the first oily phase can be characterized by five solution parameters: four Hansen solubility parameters (δ_{d}, δₚ, δₕ and δ_{T}), and Log (K_{ow}).

The Hansen solubility parameters (δ_{d}, δₚ, δₕ and δ_{T}) allow predicting whether one substance will be soluble in another.

δ_{d} is a contribution due to dispersion forces, δₚ is a contribution due to polar forces, & is a hydrogen bond contribution. These parameters are defined as follows:

$${δ}_{d} = {( \frac{Δ {E}_{d}}{V} )}^{1 / 2} \\ {δ}_{p} = {( \frac{Δ {E}_{p}}{V} )}^{1 / 2} \\ {δ}_{h} = {( \frac{Δ {E}_{h}}{V} )}^{1 / 2}$$

Here, ΔE corresponds to the vaporization energy and V the molar volume of the liquid.

δ_{T} is the solubility parameter due to the contribution of each of these parameters:

δ_{T}=(δ_{d}²+δₚ²+δₕ²)^{1/2}

Log (K_{ow}) is a measurement of the differential solubility of a chemical compound in two solvents (octanol and water) and corresponds to the octanol/water partition coefficient.

The Applicant has now discovered that it is possible to produce a multiphase composition comprising at least two oily phases, by implementing a first polar oily phase having solubility parameters δₚ of between 6 and 7.8 and δₕ between 3.7 and 10.1, at room temperature, and a second oily phase having different δₚ and δₕ solubility parameters at room temperature than those of the first oily phase.

In one particular embodiment, the polar solvent or solvents of the first oily phase according to this invention have a δₚ value of between 6 and 7.8 at room temperature.

In one particular embodiment, the polar solvent or solvents of the first oily phase according to this invention have a δₕ value of between 3.7 and 10.1 at room temperature.

In one particular embodiment, the polar solvent or solvents of the first oily phase have a δ_{d} solubility parameter of between 16 and 19 at room temperature.

The values of the various solubility parameters of the solvents may for example be calculated using the HSPiP software (for example available at the site https://www.hansen-solubility.com/buy-HSPiP-software.php). This software calculates the Hansen solubility parameters (δ_{d}, δₚ, δₕ and δ_{T}) of each solvent, based on the C8 to C18 fatty acid composition of said solvent. On the basis of the known values of the Hansen solubility parameters (δ_{d}, δₚ, δₕ and δ_{T}) listed in Table 1, the software can calculate solvent-specific parameters according to the mass proportion of each fatty acid in the solvent under consideration:

TABLE 1

| INCI name | δd | δp | δh |
|---|---|---|---|
| Caprylic acid C8:0 | 16.3 | 5.2 | 10.1 |
| Capric acid C10:0 | 16.3 | 4.2 | 8.6 |
| Lauric acid C12:0 | 16.3 | 4 | 7.5 |
| Myristic acid C14:0 | 16.3 | 3.3 | 6.6 |
| Palmitic acid C16:0 | 16.2 | 3.3 | 5.8 |
| Margaric acid C17:0 | 16.2 | 2.8 | 5.4 |
| Stearic acid C18:0 | 16.2 | 2.8 | 5.2 |
| Arachidic acid C20:0 | 16.1 | 2.7 | 4.7 |
| Behenic acid C22:0 | 16.2 | 2.3 | 4.3 |
| Lignoceric acid C24:0 | 16.1 | 2.4 | 3.9 |
| Palmitoleic acid C16:1 | 16.5 | 3.4 | 6.3 |
| Oleic acid C18:1 | 16.4 | 3 | 5.5 |
| Gadoleic acid C20:1 | 16.4 | 2.9 | 5.1 |
| Erucic acid C22:1 | 16.3 | 2.6 | 4.5 |
| Linoleic acid C18:2 | 16.7 | 3.1 | 6.1 |
| Linolenic acid C18:3 | 16.9 | 3.3 | 5.8 |
| Ricinoleic acid | 16.5 | 4.4 | 8.2 |

The HSPiP software bases its calculation, for example, on the Stefanis-Panayiotou method, for example described in the article by Stefanis, E., Panayiotou, C.: Prediction of Hansen Solubility Parameters with a New Group-Contribution Method. Int J Thermophys 29, 568-585 (2008). The Hoy and Van Krevelen method or the automated Y-MB (Yamamoto-Molecule Breaking) method may also be considered.

When the first oily phase comprises a mixture of solvents, the solubility parameters are calculated according to the mass proportion of each fatty acid in the medium. In addition, the polar solvent or solvents of the first oily phase have a surface tension greater than 30 mN/m, more preferably greater than 35 mN/m, and even more preferably greater than 40 mN/m.

The surface tension can be predicted by the HSPiP software.

In one particular embodiment, the polar solvent or solvents of the first oily phase have a log(K_{ow}) value of between 0.86 and 4.37 at room temperature.

In one particular embodiment, the polar solvent or solvents of the first oily phase are selected among dipropylene glycol dibenzoate, propylene glycol dibenzoate, triethyl citrate, castor oil, or a mixture thereof. These polar solvents have the solubility parameters shown in Table 2 below:

TABLE 2

| | | | | Surface |
|---|---|---|---|---|
| | | | | tension |
| INCI name | δd | δp | δh | (mN/m) |
| Dipropylene Glycol | 18 | 6.2 | 3.7 | 51.60 |
| Dibenzoate | | | | |
| Propylene Glycol | 18.4 | 6.5 | 3.7 | 52.50 |
| Dibenzoate | | | | |
| Triethyl citrate | 16.8 | 6.0 | 10.1 | 42.00 |
| Castor oil | 18.2 | 7.8 | 9.3 | 44.49 |

The polar solvent of the first oily phase may represent from 80% to 100% by weight, and preferably from 95% to 99.9% by weight, of said first oily phase.

The polar solvent of the first oily phase may be present, in the composition of the invention, in a proportion ranging from 5 to 50% by weight, and preferably from 8% to 25% by weight, relative to the total weight of the composition.

In addition to the polar solvent or solvents, it is possible to add to the first oily phase a moderately polar oil such as argan oil, meadowfoam seed oil, jojoba oil, or a mixture thereof, provided that said oily phase still has a surface tension greater than 30 mN/m and solubility parameters δₚ of between 6 and 7.8 and δₕ of between 3.7 and 10.1, at room temperature.

These moderately polar oils have the solubility parameters presented in the following table 3:

TABLE 3

| | | | | Surface |
|---|---|---|---|---|
| | | | | tension |
| INCI name | δd | δρ | δh | (mN/m) |
| Argan oil | 16.5 | 3.0 | 5.7 | 33.50 |
| Meadowfoam seed oil | 16.4 | 2.8 | 5.0 | 35.20 |
| Jojoba oil | 16.6 | 3.1 | 5.3 | 35.33 |

Second Oily Phase

The composition according to the invention makes use of a second oily phase, immiscible with the first oily phase, characterized by a surface tension of less than 28 mN/m, and having different δₚ and δₕ solubility parameters at room temperature than those of the first oily phase. In particular, the second oily phase has the solubility parameters δₚ of between 0 and 1, δₕ, of between 0 and 1, and/or δ_{d} of between 15.6 and 15.9, at room temperature.

In particular, the second apolar oily phase comprises one or more apolar solvents.

In one particular embodiment, the apolar solvent or solvents of the second oily phase according to the invention have a δₚ value of between 0 and 1 at room temperature, preferably between 0.01 and 0.5, and more preferably 0.1.

In one particular embodiment, the apolar solvent or solvents of the second oily phase according to the invention have a δₕ value of between 0 and 1 at room temperature, preferably between 0.01 and 0.5, and more preferably 0.1.

In one particular embodiment, the apolar solvent or solvents of the second oily phase have a δ_{d} solubility parameter at room temperature of between 15.6 and 15.9.

In addition, the apolar solvent or solvents of the second oily phase have a surface tension of less than 28 mN/m.

In one particular embodiment, the apolar solvent or solvents of the second oily phase have a log(K_{ow}) value of between 6.3 and 27.7 at room temperature.

In one particular embodiment, the apolar solvent or solvents of the second oily phase are selected among hydrocarbons, polymers obtained from a hydrocarbon monomer, or a mixture thereof.

The hydrocarbons or the polymers obtained from a hydrocarbon monomer are preferably selected among isohexadecane, isododecane, undecane, tridecane, polyisobutene, squalane, or a mixture thereof.

These apolar solvents have the solubility parameters shown in Table 4 below:

TABLE 4

| | | | | Surface |
|---|---|---|---|---|
| | | | | tension |
| INCI name | δd | δp | δh | (mN/m) |
| UNDECANE | 15.7 | 0.1 | 0.1 | 23.6 |
| TRIDECANE | 15.6 | 0.1 | 0.1 | 25.7 |
| CETIOL ULTIMATE | 15.6 | 0.1 | 0.1 | 23.75 |
| (Undecane + Tridecane) | | | | |
| SQUALANE | 15.9 | 0.1 | 0.1 | 27.65 |
| ISOHEXADECANE | 15.7 | 0.1 | 0.1 | 27.1 |
| ISODODECANE | 15.6 | 0.1 | 0.1 | 24.3 |

The apolar solvent or solvents of the second oily phase may represent from 80% to 100% by weight, and preferably from 95% to 99.9% by weight, of said first oily phase.

The apolar solvent or solvents of the second oily phase may be present in the composition of the invention in a proportion ranging from 1 to 50% by weight, and preferably from 15% to 25% by weight, relative to the total weight of the composition.

In addition to the apolar solvent or solvents, it is possible to add, to the second oily phase, natural oils of moderate polarity described above, such as argan oil, meadowfoam seed oil, jojoba oil, or a mixture thereof, provided that said oily phase still has a surface tension of less than 28 mN/m, and different δₚ and δₕ solubility parameters at room temperature than those of the first oily phase.

In a preferred embodiment, the solubility parameters at room temperature of the polar and apolar solvents respectively used in the first and second oily phases are such that

$$\sqrt{\begin{matrix}{( δ dpolar - δ dapolar )}^{2} + {( δ ppolar - δ papolar )}^{2} + \\ {( δ hpolar - δ h2 apolar )}^{2}\end{matrix}}$$

is greater than 3.5.

This characteristic makes it possible to guarantee the immiscibility of the two oily phases. Preferably, the solubility parameters at room temperature of the polar and apolar solvents respectively used in the first and second oily phases are such that

$$\sqrt{\begin{matrix}{( δ dpolar - δ dapolar )}^{2} + {( δ ppolar - δ papolar )}^{2} + \\ {( δ hpolar - δ h 2 apolar )}^{2}\end{matrix}}$$

is greater than 3.8.

Aqueous Phase

According to a preferred embodiment, the composition according to the invention comprises a third aqueous phase, immiscible with the first and second oily phases.

The aqueous phase of the composition according to the invention comprises water and any water-soluble or water-dispersible additive.

The water used may be sterile demineralized water and/or floral water such as rose water, cornflower water, chamomile water, or linden water, and/or natural thermal or mineral water, such as: Vittel water, water from the Vichy basin, Uriage water, Roche Posay water, Bourboule water, Enghien-les-Bains water, Saint Gervais-les-Bains water, Neris-les-Bains water, Allevar-les-Bains water, Digne water, Maizibres water, Neyrac-les-Bains water, Lons-le-Saunier water, Eaux Bonnes water, Rochefort water, Saint Christau water, Fumades water, and Tercis-les-bains water, Avone water.

As water-soluble additives, mention may be made in particular of polyols such as propylene glycol, butylene glycol, pentanediol, hexylene glycol, polyethylene glycols, neopentyl glycol, glycerol. The polyols may be present in an amount ranging from 0.01 to 10% by weight, preferably from 0.05 to 8% by weight relative to the total weight of the composition. According to a preferred embodiment of the invention, the composition comprises at least one polyol, preferably glycerin (glycerol).

The water-soluble additive may also be a salt, for example such as sodium chloride, which is used as an adjuvant in a composition for eye makeup removal so that the composition has an osmotic pressure close to that of tears. The presence of salt makes it possible to obtain a clear aqueous phase. The amount of salt(s) may range, for example, from 0.01 to 3% by weight and preferably from 0.05 to 2% by weight and better still from 0.1 to 1% by weight relative to the total weight of the composition.

Cosmetic Active Ingredient

The composition according to the invention may also contain conventional cosmetic adjuvants or additives which will be in one phase or the other depending on their hydrophilic or lipophilic, polar or apolar nature.

The cosmetic active ingredients may be selected among vitamins, antioxidants, moisturizing agents, anti-pollution agents, keratolytic agents, astringents, anti-inflammatories, whitening agents, microcirculation-promoting agents, dyes, softening agents, buffers, UV filters (or sunscreens), fragrances, pH adjusters (for example citric acid or sodium hydroxide), and mixtures thereof.

Examples of vitamins include vitamins A, B1, B2, B6, C, and E, and their derivatives, pantothenic acid and its derivatives, and biotin.

Examples of antioxidants include ascorbic acid and its derivatives such as ascorbyl palmitate, ascorbyl tetraisopalmitate, ascorbyl glucoside, magnesium ascorbyl phosphate, sodium ascorbyl phosphate, and ascorbyl sorbate; tocopherol and its derivatives such as tocopherol acetate, tocopherol sorbate, and other tocopherol esters; BHT and BHA; esters of gallic acid, phosphoric acid, citric acid, maleic acid, malonic acid, succinic acid, fumaric acid, cephalin, hexametaphosphate, phytic acid, and plant extracts, for example from roots of Zingiber Officinale (Ginger) such as Blue Malagasy Ginger marketed by the BIOLANDES company, from Chondrus crispus, Rhodiola, Thermus thermophilus, yerba mate leaf, oak wood, Kayu Rapet bark, Japanese Sakura cherry leaves, and ylang ylang leaves.

Examples of moisturizing agents include polyethylene glycol, propylene glycol, dipropylene glycol, glycerin, butylene glycol, xylitol, sorbitol, maltitol, mucopolysaccharides, such as chondroitin sulfuric acid, high or low molecular weight hyaluronic acid or alternatively hyaluronic acid potentiated by a silanol derivative such as the active ingredient Epidermosil® marketed by the Exymol company, and mucoitinsulphuric acid; caronic acid; atelo collagen; cholesteryl-12-hydroxystearate; bile salts, a main component of NMF (natural moisturizing factor) such as a salt of pyrrolidone carboxylic acid and a salt of lactic acid, an analogue of amino acid such as urea, cysteine, and serine; short chain soluble collagen, diglycerin PPGs, homo- and copolymers of 2-methacryloyloxyethyl phosphorylcholine such as Lipidure HM and Lipidure PMB from NOF; allantoin; glycerin derivatives such as PEG/PPG/Polybutylene Glycol-8/5/3 Glycerin from NOF sold under the trade name Wilbride®S-753 or alternatively Glyceryl Polymethacrylate from Sederma sold under the trade name Lubrajel® MS; Trimethylglycine sold under the trade name Aminocoat® by the Asahi Kasei Chemicals company, and various plant extracts such as extracts of Castanea sativa, hydrolyzed hazelnut proteins, Polianthes tuberose polysaccharide, Argania spinosa kernel oil, and mother-of-pearl extracts containing conchiolin which are sold in particular by the Maruzen company (Japan) under the trade name Pearl Extract®.

Other examples of moisturizing agents include compounds that stimulate the expression of matriptase/MT-SP1, such as carob pulp extract, as well as agents that stimulate the expression of CERT, ARNT2, or FN3K or FN3K RP; agents increasing the proliferation or differentiation of keratinocytes, either directly or indirectly by stimulating, for example, the production of β-endorphins, such as extracts of Thermus thermophilus or of Theobroma cacao bean shells, water-soluble extracts of corn, peptide extracts of Voandzeia subterranean, and niacinamide; epidermal lipids and agents increasing the synthesis of epidermal lipids, either directly or by stimulating certain β-glucosidases which modulate the deglycosylation of lipid precursors such as glucosylceramide into ceramides, such as phospholipids, ceramides, lupine protein hydrolysates, and dihydrojasmonic acid derivatives.

Examples of anti-pollution agents include Moringa pterygosperma seed extract (for example Purisoft® from LSN); shea butter extract (for example Detoxyl® from Silab); a mixture of ivy extract, phytic acid, sunflower seed extract (for example Osmopur® from Sederma).

Examples of keratolytic agents include α-hydroxy acids (for example glycolic, lactic, citric, malic, mandelic, or tartaric acids) and β-hydroxy acids (for example salicylic acid), and their esters, such as C12-13 alkyl lactates, and plant extracts containing these hydroxy acids, such as Hibiscus sabdariffa extracts.

Examples of astringents include witch hazel extracts.

Examples of anti-inflammatory agents include bisabolol, allantoin, tranexamic acid, zinc oxide, sulfur oxide and its derivatives, chondroitin sulfate, glycyrrhizic acid and its derivatives such as glycyrrhizinates.

Examples of wightening agents include arbutin and its derivatives, ferulic acid (such as Cytovector®: water, glycol, lecithin, ferulic acid, hydroxyethylcellulose, marketed by BASF) and its derivatives, kojic acid, resorcinol, lipoic acid and its derivatives such as resveratrol diacetate monolipoate as described in patent application WO2006134282, ellagic acid, leucodopachrome and its derivatives, vitamin B3, linoleic acid and its derivatives, ceramides and their homologues, a peptide as described in patent application WO2009010356, a bioprecursor as described in patent application WO2006134282 or a tranexamate salt such as cetyl tranexamate hydrochloride salt, a licorice extract (extract of Glycyrrhiza glabra) which is sold in particular by the Maruzen company under the trade name Licorice extract®, a whitening agent that also has an antioxidant effect, such as vitamin C compounds, including ascorbate salts, ascorbyl fatty acid esters, or sorbic acid, and other derivatives of ascorbic acid, for example ascorbyl phosphates, such as magnesium ascorbyl phosphate and sodium ascorbyl phosphate, or saccharide esters of ascorbic acid, which include, for example, ascorbyl-2-glucoside, 2-O-alpha-D-glucopyranosyl-L-ascorbic acid, or 6-O-beta-D-galactopyranosyl. An active agent of this type is sold in particular by the company DKSH under the trade name Ascorbyl glucoside®.

Examples of microcirculation-promoting agents include extract from lupine (such as Eclaline® from Silab), from butchers broom, from horse chestnut, from ivy, from ginseng, or from sweet clover, caffeine, nicotinate and its derivatives, an extract of Corallina officinalis algae such as the one marketed by CODIF; and mixtures thereof. These agents promoting cutaneous microcirculation can be used to prevent dullness of the complexion and/or to improve uniformity and radiance of the complexion.

Among the softening agents, mention may be made in particular of allantoin, bisabolol, plankton, and certain plant extracts such as rose extracts and sweet clover extracts.

According to a preferred embodiment, the cosmetic active ingredient is a moisturizing active ingredient. According to an even more preferred embodiment, the moisturizing active ingredient is hyaluronic acid.

The composition according to the invention may comprise from 0.0001 to 10% by weight of cosmetic active ingredient, preferably from 0.001 to 5% by weight, and more preferably from 0.002 to 1% by weight, relative to the total weight of the aqueous phase.

Preferably, the composition according to the invention has a pH of between 3.5 and 7.5.

The compositions according to the invention are preferably free of silicones, fluorinated derivatives, polyethylene, or polypropylene glycols. The term “free of silicone, fluorinated derivatives, polyethylene, or polypropylene glycols” is understood to mean that the composition comprises less than 1% by weight of each of these compounds, preferably less than 0.5%.

In addition, the compositions according to the invention are free of mono-alcohols, i.e. it comprises less than 3% of them by weight, preferably less than 2% by weight, more preferably less than 1% by weight, and more preferably less than 0.5% by weight. Alcohols are usually used in the aqueous phase to impart a cooling effect to cosmetic compositions. However, they have a drying effect on the skin and poor tolerance for sensitive skin. The inventors have observed that it is possible to provide alcohol-free compositions having a cooling effect, by means of the compositions according to the invention.

The compositions described above may be packaged, in known manner, in a bottle having a single compartment. The user must then shake the bottle before pouring the contents onto a cotton ball. Provision may also be made for the different phases of the composition to be introduced into two independent compartments of a same bottle, a system being provided for mixing them at the time of distribution.

The composition according to the invention may be used for any topical application; in particular, it may constitute a cosmetic or dermatological composition. It may in particular be used for caring for, cleaning, and/or removing makeup from the skin, eyes, and/or skin appendages.

Another object of the invention is a skincare product, characterized in that it comprises a composition as defined above.

According to a preferred embodiment of the invention, said product may be an anti-aging serum, preferably having an exfoliating effect, or a makeup remover for the face, in particular for the eyes and/or lips.

Another object of the invention is a skincare process, consisting of applying to the skin a composition as defined above. In particular, this may be a cosmetic process of removing makeup from, cleaning, and/or caring for the skin, eyes, and/or skin appendages, comprising the application of a cosmetic composition as defined above, to the skin, eyes, and/or skin appendages.

The invention also relates to a method of preparing a composition as defined above, comprising the following steps:
cold preparation of each of the phases by mixing the ingredients with a blender,
introduction of the second apolar oily phase into the first polar oily phase.

When the composition comprises an aqueous phase, the method involves introducing the mixture of apolar and polar oily phases into the aqueous phase.

### BRIEF DESCRIPTION OF DRAWINGS

FIG. 1 illustrates the method of preparing a composition according to the invention.

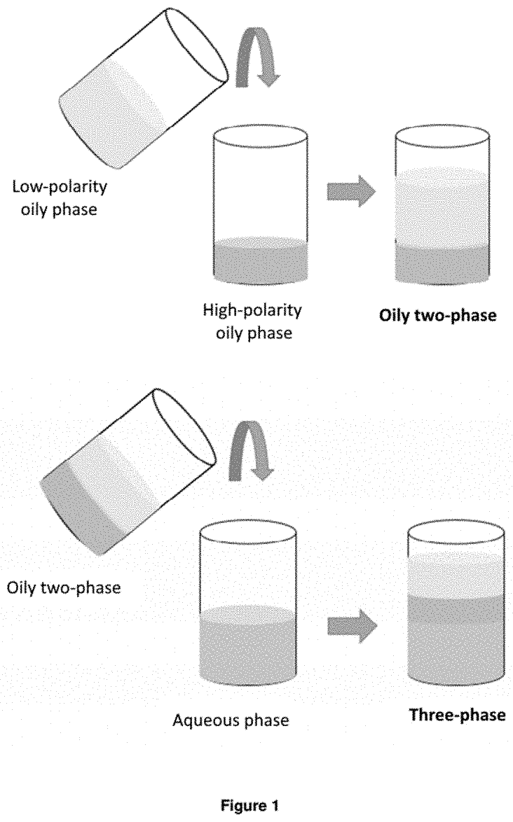

The following examples of compositions according to the invention are provided for illustrative purposes and are not limiting. The quantities are given as % by weight unless otherwise stated.

### EXAMPLES

### Example 1: Anti-Aging Serum

A three-phase composition having the following composition was prepared.

TABLE 5

| | | Content |
|---|---|---|
| | INCI name | (% by weight) |
| Aqueous | Demineralized water | 41.860% |
| phase | NATURAL AHA COMPLEX | 10.000% |
| | Chlorhexidine digluconate | 0.22% |
| | Water-soluble active ingredient | 4.04% |
| | Glycerin | 3.00% |
| | Butylene glycol | 4.000% |
| | Dye | 0.03% |
| | L-ARGININE | 2.75% |
| First oily | Castor oil | 15.00% |
| phase | Fragrance | 0.10% |
| Second oily | Squalane | 9.00% |
| phase | CETIOL ULTIMATE | 10.00% |
| | (undecane & tridecane) | |

Each phase was prepared cold, separately, by mixing the ingredients of each phase using a magnetic stir bar. The second oily phase was then introduced into the first oily phase, then the mixture was introduced into the aqueous phase.

The serum thus obtained is in the form of a composition which visually has three distinct immiscible phases. It has an exfoliating/peeling effect when applied to the skin.

### Example 2: Facial Makeup Remover

A three-phase composition having the following composition was prepared.

TABLE 6

| | | Content |
|---|---|---|
| | INCI name | (% by weight) |
| Aqueous | Demineralized water | 64.578% |
| phase | Chlorhexidine digluconate | 0.220% |
| | Glycerin | 4.000% |
| | P extract G (active ingredient) | 0.200% |
| | L-ARGININE | 0.002% |
| First oily | Castor oil | 10.000% |
| phase | Active | 0.100% |
| Second oily | Cetiol Ultimate | 6.000% |
| phase | (undecane and tridecane) | |
| | Squalane | 9.000% |
| Additional | Limnanthes Alba Seed Oil | 7.000% |
| oils | Jojoba oil, color removed | 10.000% |

Each phase was prepared cold, separately, by mixing the ingredients of each phase using a magnetic stir bar. The second oily phase was then introduced into the first oily phase, then the mixture was introduced into the aqueous phase.

The facial makeup remover thus obtained takes the form of a composition visually having three very distinct immiscible phases. It gently removes makeup from the skin, eyes, and lips.

1-16. (canceled)

17. A multiphase cosmetic composition comprising:
a first polar oily phase characterized by a surface tension greater than 30 mN/m, and having the following solubility parameters at room temperature: δₚ of between 6 and 7.8 and δₕ of between 3.7 and 10.1,
a second apolar oily phase, immiscible with the first oily phase, characterized by a surface tension of less than 28 mN/m, and having different δₚ and δₕ solubility parameters at room temperature than those of the polar solvent used in the first oily phase,
said composition being surfactant-free.

18. The composition according to claim 17, wherein the polar solvent or solvents used in the first oily phase have a Sa solubility parameter at room temperature of between 16 and 19.

19. The composition according to claim 17, wherein the apolar solvent or solvents used in the second oily phase have the following solubility parameters at room temperature:
δₚ of between 0 and 1, preferably between 0.01 and 0.5, and more preferably 0.1
δₕ of between 0 and 1, preferably between 0.01 and 0.5, and more preferably 0.1 and
δ_{d} of between 15.6 and 15.9.

20. The composition according to claim 17, wherein the solubility parameters at room temperature of the polar and apolar solvents are such that
$$\sqrt{\begin{matrix}{( δ dpolar - δ dapolar )}^{2} + {( δ ppolar - δ papolar )}^{2} + \\ {( δ hpolar - δ h 2 apolar )}^{2}\end{matrix}}$$
is greater than 3.5.

21. The composition according to claim 17, wherein the polar solvent or solvents of the first oily phase are selected among dipropylene glycol dibenzoate, propylene glycol dibenzoate, triethyl citrate, castor oil, or a mixture thereof.

22. The composition according to claim 17, wherein the apolar solvent or solvents of the second oily phase are selected among hydrocarbons, polymers obtained from a hydrocarbon monomer, or a mixture thereof.

23. The composition according to claim 22, wherein the hydrocarbons or the polymers obtained from a hydrocarbon monomer are selected among isohexadecane, isododecane, undecane, tridecane, polyisobutene, squalane, or a mixture thereof.

24. The composition according to claim 17, comprising natural oils of moderate polarities such as argan oil, meadowfoam seed oil, jojoba oil, or a mixture thereof, subject to not modifying the δₚ and δₕ solubility parameters at room temperature of the first and second oily phases.

25. The composition according to claim 17, comprising a third aqueous phase, immiscible with the first and second oily phases.

26. The composition according to claim 23, wherein the aqueous phase comprises water, and optionally at least one polyol and/or at least one salt.

27. The composition according to claim 24, wherein the polyol is selected among propylene glycol, butylene glycol, pentanediol, hexylene glycol, polyethylene glycols, neopentyl glycol, glycerol, and preferably the polyol is glycerol.

28. The composition according to claim 17, comprising a cosmetic active ingredient, preferably selected among vitamins, antioxidants, moisturizing agents, anti-pollution agents, keratolytic agents, astringents, anti-inflammatories, wightening agents, microcirculation-promoting agents, dyes, softening agents, buffers, UV filters (or sunscreens), fragrances, pH adjusters (for example citric acid or sodium hydroxide), and mixtures thereof.

29. A multiphase cosmetic composition comprising:
a first polar oily phase comprising dipropylene glycol dibenzoate, propylene glycol dibenzoate, triethyl citrate, castor oil, or a mixture thereof,
a second apolar oily phase, immiscible with the first oily phase, comprising hydrocarbons, polymers obtained from a hydrocarbon monomer, or a mixture thereof,
said composition being surfactant-free.

30. A skincare product, comprising a composition as defined in claim 17.

31. The skincare product according to claim 30, wherein it constitutes an anti-aging serum, preferably having an exfoliating effect, or a makeup remover for the face, in particular for the eyes and/or lips.

32. A skin care process, consisting of applying to the skin a composition as defined claim 17 and/or a skincare product comprising said composition.
